(19) United States
(12) Patent Application Publication
(10) Pub. No.: US 2006/0241663 A1
(43) Pub. Date: Oct. 26, 2006

(54) Surgical cutter

(75) Inventors: Mark W. Rice, Minneapolis, MN (US); Michael E. Lancial, St. Louis Park, MN (US); John F. Otte, St. Anthony, MN (US)

Correspondence Address: WOOD, HERRON & EVANS;ZIMMER SPINE, 2700 CAREW TOWER, 441 VINE STREET, CINCINNATI, OH, 45202, US

(73) Assignee: Zimmer Spine, Inc., Minneapolis, MN (US)

(21) Appl. No.: 11/111,369

(22) Filed: Apr. 21, 2005

(51) Int. Cl. A61B 17/32

(52) U.S. Cl. 606/167

(57) ABSTRACT

The invention provides a device and method for cutting an object inside a surgical wound. In one embodiment of the invention, a cutter comprises an elongated body having a distal portion and a proximal portion; a blade holder disposed at least partially in the distal portion and adapted to secure a blade having a cutting edge, the blade holder being movable between a withdrawn position and a advanced position; a blade shield positioned to cover substantially the entire cutting edge when the blade holder is in the withdrawn position and expose at least a portion of the cutting edge when the blade holder is in the advanced position; and a stop disposed in the distal portion and adapted to stabilize an object to be cut against the cutting edge while the blade holder moves from the withdrawn position toward the advanced position.

### FIELD OF THE INVENTION

The invention relates generally to surgical cutters. More particularly, the invention relates to a cutter for severing an object inside an incision while protecting other material surrounding the cutter.

### BACKGROUND OF THE INVENTION

In surgery, a variety of specialized surgical instruments are typically used to carry out the operations. Special-purpose instruments should preferably facilitate safe, precise and efficient surgical procedures. Such instruments exist for various procedures in surgeries, including spinal surgeries. There remain, however, needs for additional types of instruments or improved instruments for new and existing procedures.

For example, surgical procedures frequently call for cutting of objects inside surgical wounds. Objects to be cut includes implanted components such as cords and rods, threads, tissues and bones. The invention disclosed herein is aimed at providing a cutter that provides safe, precise and efficient cutting of objects inside surgical wounds.

### SUMMARY OF THE INVENTION

Generally, the invention provides an elongated surgical cutter that can reach into surgical wounds and supports the object being cut while an advancing blade on the cutter severs the object. The cutter makes it unnecessary to hold or otherwise handle the object with another instrument or hand and thus enhances the visibility of the cutting site. The cutter further shields the cutting edge of the blade when the blade is not actuated to cut the object. In one embodiment of the invention, a cutter comprises an elongated body having a distal portion and a proximal portion; a blade holder disposed at least partially in the distal portion and adapted to secure a blade having a cutting edge, the blade holder being movable between a withdrawn position and a advanced position; a blade shield positioned to cover substantially the entire cutting edge when the blade holder is in the withdrawn position and expose at least a portion of the cutting edge when the blade holder is in the advanced position; and a stop disposed in the distal portion and adapted to stabilize an object to be cut against the cutting edge while the blade holder moves from the withdrawn position toward the advanced position.

In another embodiment of the invention, the blade holder in the above-described cutter is in sliding contact with the body and comprises: a base member in sliding contact with the body; and a switching member connected to the base member and movable between an open position and a closed position relative to the base member, wherein the blade holder is configured to permit the blade to be mounted on, and detached from, the blade holder when the switching member is in the open position and to lock the blade to the blade holder when the switching member is in the closed position. The blade holder can further include a locking member configured to selectively maintain the switching member in either the open or the closed position relative to the base member.

In another embodiment of the invention, a method of severing an object disposed in an incision in a patient, the method comprising: mounting a blade on a blade holder of a cutter, the blade comprising a cutting edge, wherein the cutter comprising: an elongated body having a distal portion and a proximal portion, a blade holder disposed at least partially in the distal portion and adapted to secure the blade, the blade holder being movable between a withdrawn position and a advanced position, a blade shield positioned to cover substantially the entire cutting edge when the blade holder is in the withdrawn position and expose at least a portion of the cutting edge when the blade holder is in the advanced position, and a stop disposed in the distal portion and adapted to stabilize an object to be cut against the cutting edge while the blade holder moves from the withdrawn position toward the advanced position. The method further comprises placing the blade holder in the withdrawn position; while maintaining the blade holder in the withdrawn position, placing the distal end of the cutter inside the incision and positioning the object between the blade and the stop; and moving the blade holder toward the advanced position to sever the object.

### BRIEF DESCRIPTION OF THE DRAWING

Other objects and advantages of the invention will become apparent upon reading the following detailed description and upon reference to the drawings in which:

FIG. 1 shows an application of a device according to one aspect of the invention;

FIG. 2 is a perspective view of a cutter in a first embodiment of the invention;

FIG. 3 is a close-up view from one side of the distal portion of the cutter shown in FIG. 2;

FIG. 3(a) is close-up view of the locking mechanism shown in FIG. 3;

FIG. 4 is a close-up view from the other side of the distal portion of the cutter shown in FIG. 2;

FIG. 5 shows a cutter in a second embodiment of the invention;

FIG. 6 shows a close-up view of the cutter in FIG. 5 in a state where the cutting blade is in a fully withdrawn position;

FIG. 7 shows a close-up view of the cutter in FIG. 5 in a state where the cutting blade is in a partially advanced position;

FIG. 8 shows a close-up view of the cutter in FIG. 5 in a state where the cutting blade is in a fully advanced position;

FIG. 9 shows the tip portion of a cutter in a third embodiment of the invention;

FIG. 10 shows the tip portion of the cutter shown in FIG. 9 viewed from another angle;

FIG. 11 shows the tip portion of a cutter in a fourth embodiment of the invention;

FIG. 12 shows the tip portion of the cutter shown in FIG. 11 viewed from another angle;

FIG. 13 shows a cutter in a fifth embodiment of the invention in an operational state (i.e., with the blade securely mounted and in the closed position);

FIG. 14 shows the cutter shown in FIG. 13, with the blade holder in the open and unlocked position;

FIG. 15 shows the cutter shown in FIG. 13, with the blade holder in the open and locked position;

FIG. 16(a) shows a disassembled view of the cutter shown in FIG. 13, showing the locking mechanism;

FIG. 16(b) shows the locking mechanism in FIG. 16(a) in closed and locked state;

FIG. 16(c) shows the locking mechanism in FIG. 16(a) in open and locked position;

FIG. 17 shows one side of the tip portion of a cutter in a sixth embodiment of the invention;

FIG. 18 shows the other side of the tip portion of the cutter shown in FIG. 17;

FIG. 19 shows the top view of the tip portion of the cutter shown in FIG. 17; and

FIG. 20 shows an object-retaining structure in the tip portion of cutter body in a seventh embodiment of the invention.

### DETAILED DESCRIPTION OF SPECIFIC EMBODIMENTS

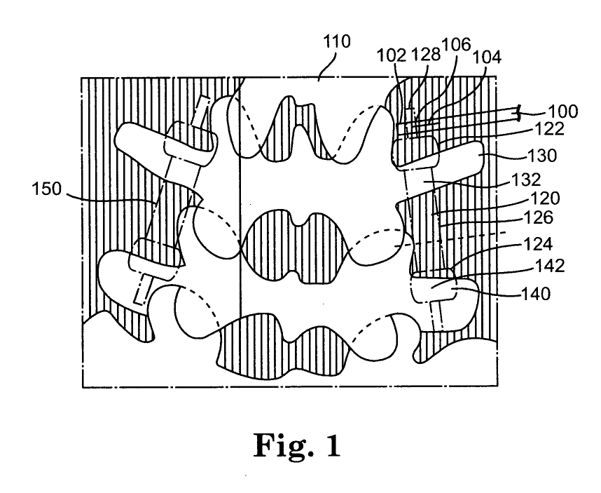

An example of the surgical situations in which a cutter 100 according to one aspect of the invention is particularly useful is illustrated in FIG. 1. A top view of the cutter 100 is shown against a posterior view of a segment of a spine 110 at least partially visible to a surgeon through one or more surgical wounds (not illustrated). A spinal stabilization device 120 is implanted to stabilize a vertebra 130 relative to another 140. Pedicle screws 122 and 124 are driven into the pedicles 132 and 142, respectively, of the vertebrae 130 and 140, respectively. A spacer 126 is positioned between the heads of the pedicle screws 122 and 124. A cord 128 is threaded through the spacer 126 and clamped in the heads of the pedicle screws 122 and 124 after being tensioned to put the spacer 126 in compression. The cutter 100, which has a stop block 102 and a sliding blade 104 and with a gap 106 having a top opening 108 between the stop block 102 and the sliding blade 104, is extended into the surgical wound while the blade 104 is retracted. The cutting edge of the blade is also shielded by the cutter 100 to protect the surrounding tissues and other materials from being accidentally cut. An excess segment of the cord 128 is then positioned inside the gap 106 through the top opening 108. The blade 104 is then advanced towards the stop block 102, which supports the cord 128 against the advancing blade 104. The excess portion of the cord 128 is thus cut from the stabilization device 120 without requiring the operator to hold or otherwise handle the excess portion of the cord 128. Similar operations are typically carried out to implant a second stabilization device 150.

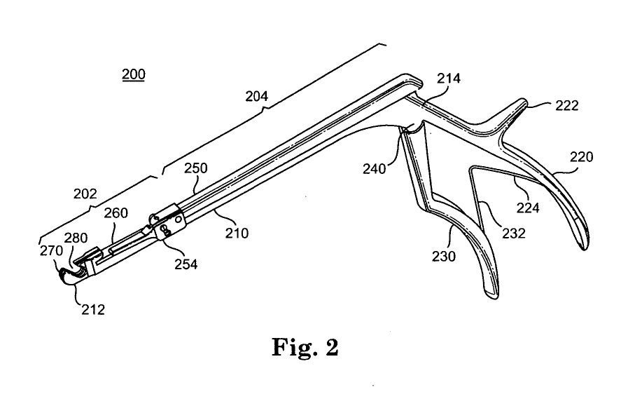

Referring to FIG. 2, a cutter 200 in a first illustrative embodiment of the invention comprises a distal portion 202 and a proximal portion 204. The cutter 200 comprises an elongated body 210 having a distal portion 212 and a proximal portion 214. In the proximal portion 204, the cutter 200 has a stationary rear handle 220 connected to the proximal portion 214, to be grasped in the palm of the surgeon's hand. A spike 222 supports the area of the hand between the thumb and index finger. A movable forward handle 230 is attached to the rear handle 220 at a pivot point 240 and is adapted to be engaged by the fingers of the surgeon to pull the forward handle 230 toward the rear handle 220. A blade holder 250 is secured to the top of the body 210 and can move relative to the body 210 in a sliding motion. The blade holder 250 is linked to the forward handle 230, which serves as an actuator that, when pulled toward the rear handle 220, slides the blade holder forward toward the distal portion 212 of the body 210 to an advanced position from a withdrawn position. A resilient member, such as a pair of leaf springs 224 and 232, is connected between the rear and forward handles 220 and 230 to bias them away from each other, thereby maintaining the blade holder in the withdrawn position when the forward handle 230 is released. Other suitable types of resilient members, such as coil springs, can also be used for this purpose.

The proximal portion 204 of the cutter 200 described above is well known in the art. For example, Kerrison-type rongeurs and similar surgical instruments have the same of similar structures as the proximal portion 204 of the cutter 200. It should be further noted that other types of actuating mechanisms than the type disclosed above, such as a manually-actuated or motor actuated worm gear system, can also be used.

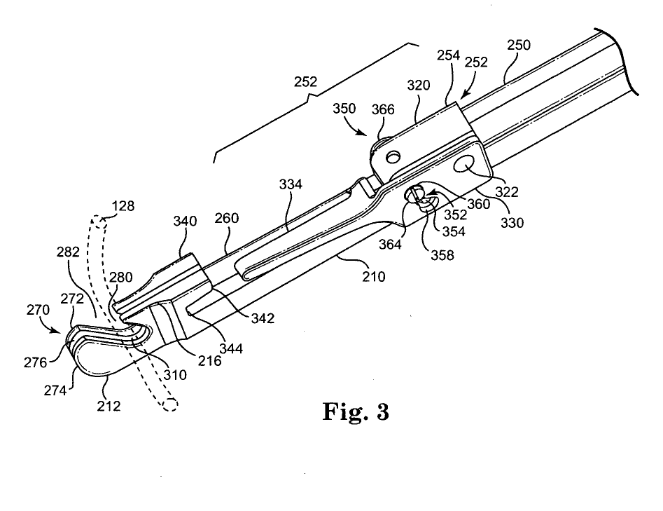

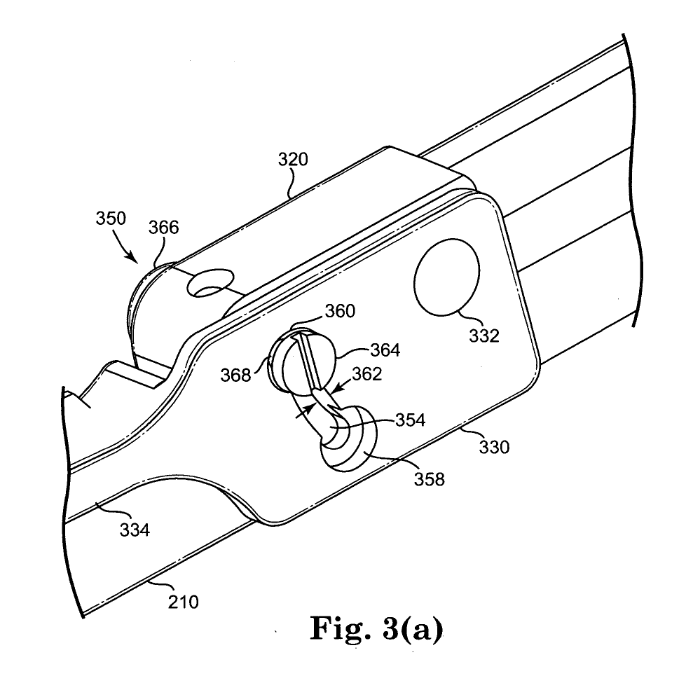

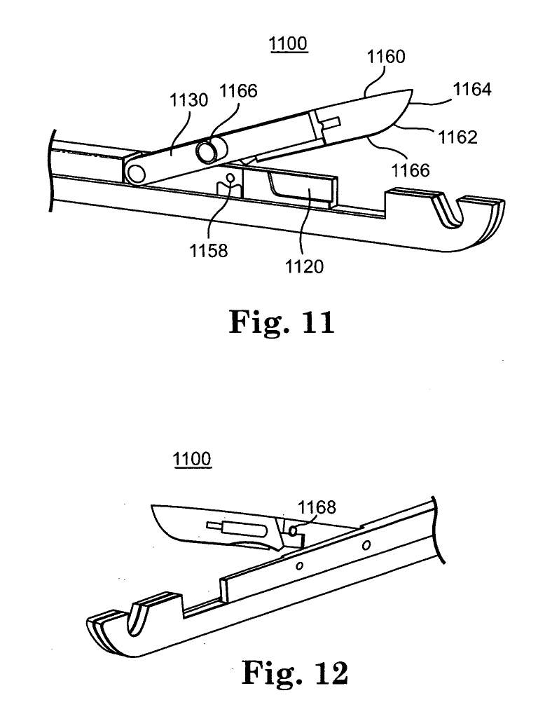

With additional reference to FIG. 3 and 4, the distal portion 202 of the cutter 200 includes the distal potion 252 of the blade holder 250, the distal portion 212 of the body 210, and a blade 260, which is mounted on the blade holder 250. The blade 260 in this illustrative embodiment is a common surgical scalpel blade, but can be other types of blades suitable for surgery. The blade 260 has a cutting edge (not shown in FIGS. 2-4 but can be seen in, among others, FIG. 11, as the cutting edge 1162 of the blade 1160), which in this illustrative embodiment has a forward portion and bottom portion (1164 and 1166, respectively, in FIG. 11). The distal portion 212 of the body 210 has a slot 310, inside which substantially all of the cutting edge of the blade 260 is positioned when the blade holder 250 is in the withdrawn position. Parts of the distal portion 212 of the body 210 thus function as a shield covering the cutting edge of the blade 260 when the blade holder is in the withdrawn position. The distal portion 212 of the body 210 further includes a stop 270, which in the illustrative embodiment comprises blocks 272 and 274, which stabilize the object to be cut (e.g., cord 128) against the advancing cutting edge of the blade 260. The stop 270 has a gap 276 between the blocks 272 and 274 to allow the forward edge of the blade 260 to pass the interface between the object to be cut and the blocks 272 and 274 to ensure complete severing of the object.

The distal portion 212 of the body 210 defines a gap 280 having a top opening 282, through which the object to be cut can be received into the gap 280 and positioned between the blade 260 and stop 270. Thus, an elongated object such as a cord 128 can be captured into the gap 280 from a direction generally transverse to the longitudinal direction of the object rather than be threaded through the gap 280.

The distal portion 202 of the cutter 200 comprises a blade mount 254 in the distal portion 252 of the blade holder. The blade mount 254 comprises a base member 320, which is in sliding contact with the body 210, and a switching member 330, which is connected to the base member 320 and movable between an open position and a closed position relative to the base member 320. In this illustrative embodiment of the invention, the switching member 330 is pivotally attached to the base member 320 via a pivot point 332. The switching member 330 further comprises a distal portion 334 overhanging the body 210. The distal portion 334 in this embodiment comprises an elongated boss 336 for attaching the blade 260 to the switching member 330.

The blade mount 254 further comprises a locking member 350 for selectively maintaining the switching member 330 in its open or closed position relative to the base member 320. With further reference to FIG. 3(a), the locking member 350 comprises a lower recess 358 and an upper recess 360 in the switching member 330. An arcuate slot 354, which is narrower than the diameters of the recesses 358 and 360 and has a depth extending through the thickness 362 of the switching member 330, connects the recesses 358 and 360. A locking pin 368 passes through the base member 320 and protrudes into the switching member 330. A disk-shaped head 364, sized to snugly fit into the recesses 358 and 360, is connected to the protruding end of the locking pin 368. A push button 366 is connected to the other end of the locking pin 368. The locking pin 368 is biased by a resilient member (not shown) inside the base member such that the push button 366 is biased away from the base member 320, and the head 364 is biased toward the switching member 330. The slot 354 generally follows an arch segment of an imaginary circle centered on the pivot point 332, such that the switching member 330 can freely rotate about the pivot point 332 through the angle spanned by the slot 354 when the head 364 is not engaged to either of the recesses 358 and 360.

In operation, the operator pushes the button 366 to disengage the head from either of the recesses 358 and 360 so that the switching member 330 can freely tilt. When either end of the slot 354 reaches the locking pin 368, the position of either of the recesses 358 and 360 is matched to that of the head 364. The operator then releases the push button 366 to allow the head 364 to be engaged in the matching recess, and the switching member 330 is locked in a corresponding position relative to the base member 320. More specifically, when the head is engaged in the lower recess 358, the switching member 330 is locked in the open position, in which the blade 260 can be mounted on, or detached from, the switching member 330. When the head 364 is engaged in the upper recess 360, the switching member 330 is locked in the closed position, in which the blade 260 is maintained in the operating position, i.e., with the cutting edge protected and ready to be pushed to cut the cord 128.

It should be noted that other configurations of the locking member 350 can also be used to achieve the desired performance. For example, the push button 366 can be on the side of the switching member 330, with the recesses 358 and 360 and slot 354 on the base member 320. Other locking mechanisms, such as spring-loaded ball bearing detents, can also be used.

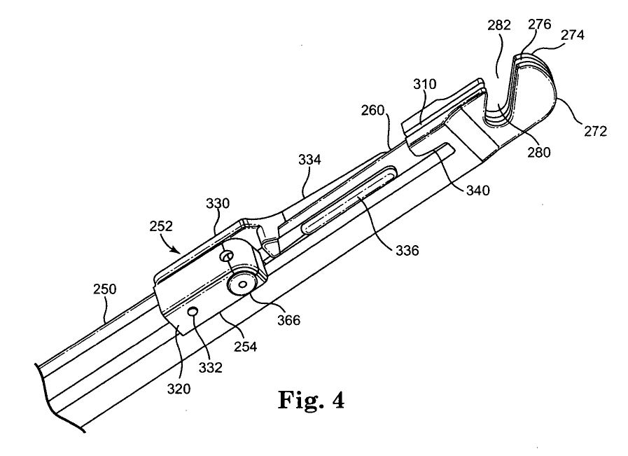

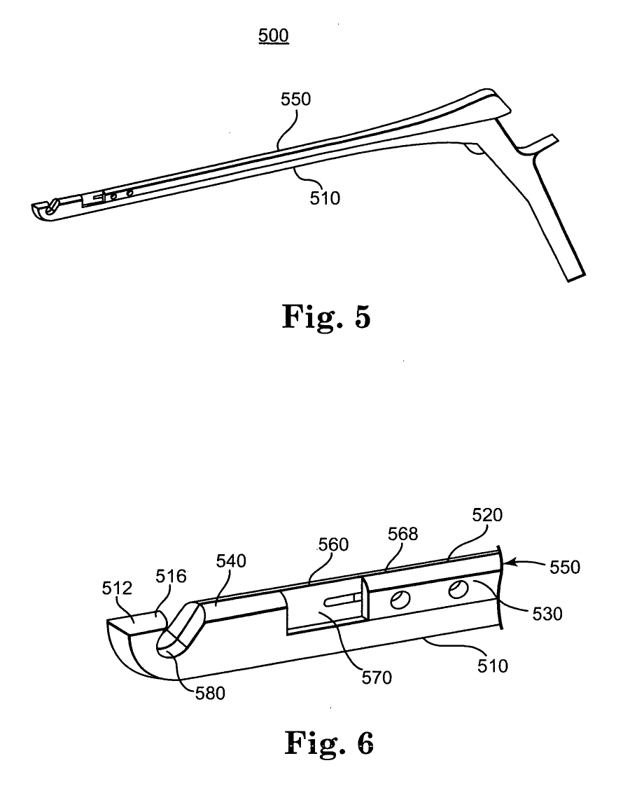

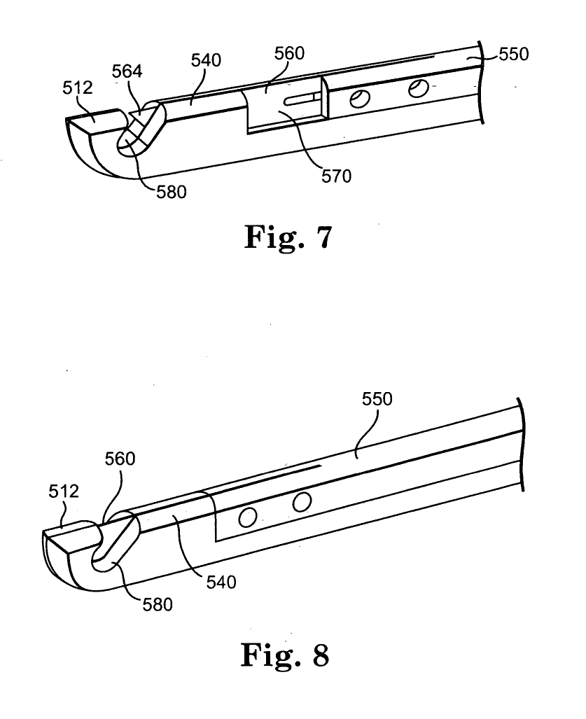

The distal portion 202 of the cutter 200 further comprises a guide member 340 overhanging the base 210. The guide member 340 includes a guide portion 342, which ramps downwardly in the direction of blade advancement. Thus, at least during the last portion of blade advancement in a cutting operation, the distal portion 334 becomes engaged under the guide member 340. The blade 260 is thus prevented from being lifted up by the resistance force from the object being cut. The distal end of the guide member 340 also serves as an end stop to prevent further advancement of the blade 260. The gap 280 in this embodiment is slanted with its top opening 282 positioned closer than the bottom to the distal end of the cutter. This configuration facilitates easy capturing of the object to be cut. Other configurations can be used to achieve desired performance. As an example, as in several embodiments illustrated below, the gap can be slanted with the top opening positioned toward the proximal end of the cutter. As another example, the gap can be an aperture on the cutter body without any side opening. The object to be cut in this case needs to be threaded through the aperture. Referring to FIGS. 5, 6, 7 and 8, in a second embodiment of the invention, a cutter 500 has an over-all design similar to that shown in FIG. 2 (the forward handle and leaf springs are not shown). In this illustrative embodiment, the base portion 568 of the blade 560 is guarded on both sides by the distal portions 520 and 530 of the blade holder 550. The top end 516 of the distal portion 512 projects toward the blade 560 so as to provide a downward component of the force supporting the object being cut, thereby more securely retaining the object in the gap 580. When the blade holder 550 is in the fully retracted state, as shown in FIG. 6, the cutting edge (not shown) of the blade 560 is covered by the cutter body 510, and the gap 580 is ready to receive the object to be cut. The end of the blade holder 550 is at a distance 570 from the portion 540 of the body 550 that protects the forward portion 564 of the blade 560 to allow the blade 560 to advance. As the blade holder is pushed toward the distal end 512 of the body 510, as shown in FIG. 7, the forward cutting edge 564 emerges from the protective portion 540 and begins to cut the object. Finally, as illustrated in FIG. 8, the end of the blade holder 550 reaches the end of its travel and is stopped by the portion 540. At this point, the forward cutting edge 564 (not shown in FIG. 8) has traversed the entire gap 580 and into the distal portion 512 of the body 510, ensuring a complete severing of the object.

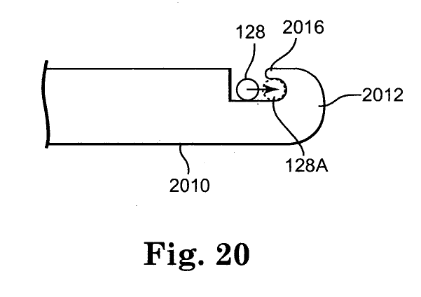

As schematically shown in FIG. 20, similar to the rearward projection of the distal portion 512, the distal portion 2012 of the cutter body 2010 in an alternative embodiment of the invention includes a overhanging portion 2016 projecting toward the proximal end of the cutter. Thus, an object to be cut 128 can be securely held in a position 128A under the overhanging portion 2016 while being cut.

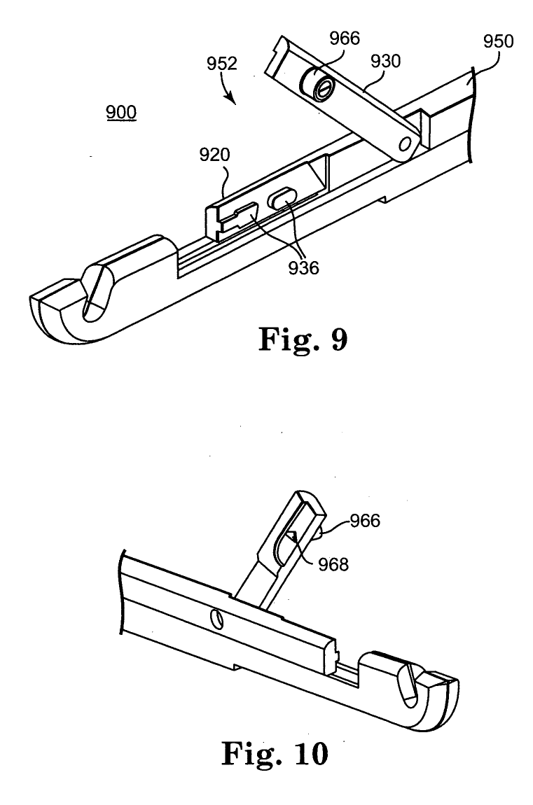

As shown in FIGS. 9 and 10, in a third embodiment of the invention, the distal portion 952 of the blade holder 950 of a cutter 900 comprises a base member 920 and a switching member 930 which is pivotally attached to the base member 920. The blade (not shown) in this case is mounted on the base member by sliding a slot on the blade over the bosses 936 on the base member 920. The switching member 930 is lifted to permit mounting or removal of the blade and closed down to shield the base of the blade after the blade is installed. A spring-loaded ball detent 966 is mounted on the switching member 930, with a ball bearing 968 biased toward the base member 920. A recess, such as a portion of the slot on the blade or dimple formed on the base member 920 is positioned to be engaged to the ball bearing 968 when the switching member 930 is in the closed and maintain the switching member 930 in the closed position.

With reference to FIGS. 11 and 12, in a fourth embodiment of the invention, a cutter 1100 is similar to the cutter 900 shown in FIGS. 9 and 10 but with the blade 1160 mounted on the switching member 1130 instead of the base member 1120. In this case, a spring-loaded ball detent 1166 is mounted on the switching member 1130, with a recess 1158 on the base member 1120 to engage the ball bearing 1168 in the detent 1166 to secure the switching member 1130 in the closed position.

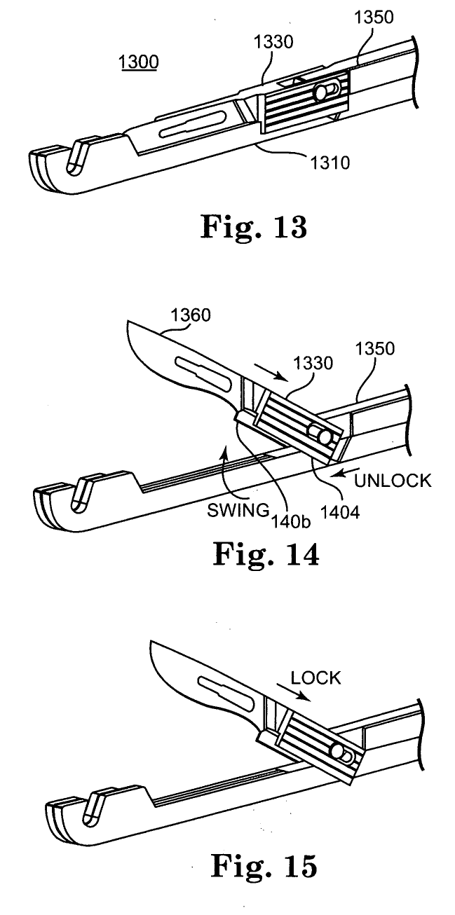

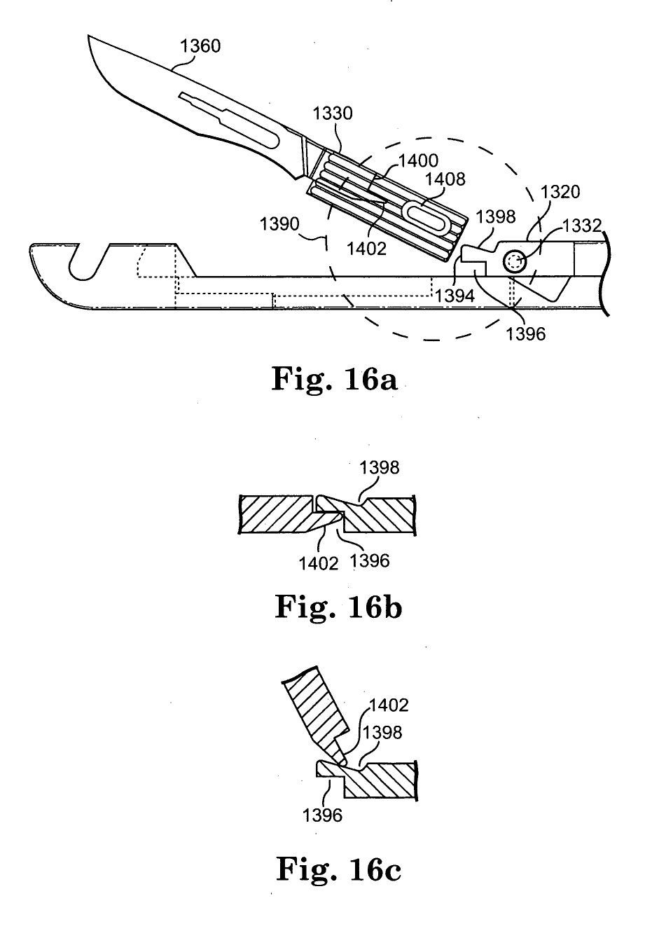

Referring to FIGS. 13, 14, 15, 16(a), 16(b) and 16(c), a cutter 1300 in a fifth embodiment of the invention includes a pivotal blade mount 1330 and a locking member 1390 that is configured to selectively maintain the blade mount 1330 in either an open position (FIG. 15) or a closed position (FIG. 13). The blade holder 1350 comprises a switching member 1330, which accepts a blade 1360 and is attached to the remaining portions of the blade holder 1350 at a pivot pin 1332. The switching member 1330 comprises a middle potion 1400 and two side panels 1404 and 1406. Each side panel has an elongated slot (only the slot 1408 shown on side panel 1404), which is designed to engage the pivot pin 1332. The switching member 1330 can thus both tilt and slide relative to the pivot pin 1332. The locking member 1390 includes a base member 1320 at the end of the base portion of the blade holder 1350. The base member 1320 has a ledge 1394, a lower recess 1396 below the ledge 1394 and an upper recess 1398 above the ledge 1394. The middle portion 1400 has a protrusion 1402, which can be pushed into either of the recesses 1396 and 1398 when the switching member 1330 slides toward the proximal end of the cutter 1300. When the switching member 1330 slides toward the distal end of the cutter 1300, the ledge 1394 is out of the path of movement of the protrusion 1402, thereby allowing the switching member 1330 to move between the open and closed positions.

In operation, the switching member 1330 is pulled toward the distal end of the cutter 1330 and-swung to the desired position (open or closed). See, FIG. 14. The switching member 1330 is then pushed toward the proximal portion of the cutter 1330 to engage the protrusion 1402 in one of the recesses 1396 and 1398 to lock the switching member in the desired location. More specifically, the switching member 1330 is locked in the closed position (FIG. 13) when the protrusion 1402 is engaged in the lower recess 1396, and is locked in the open position (FIG. 15) when the protrusion 1402 is engaged in the upper recess 1398.

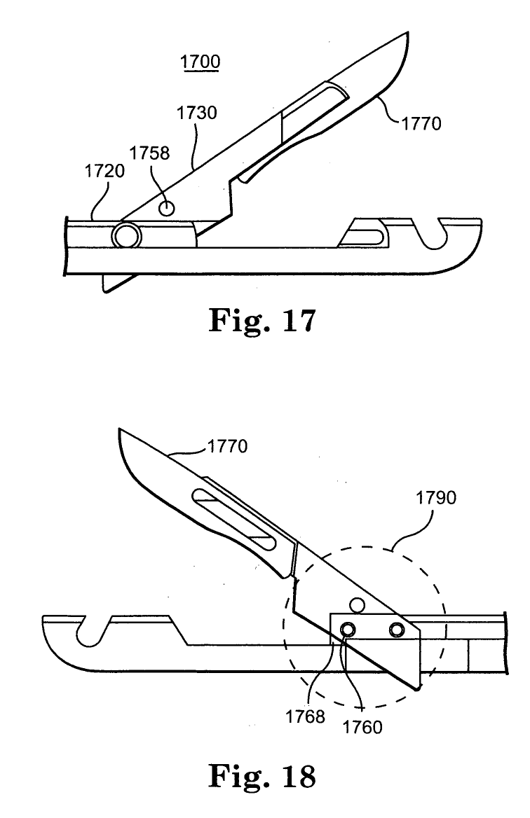

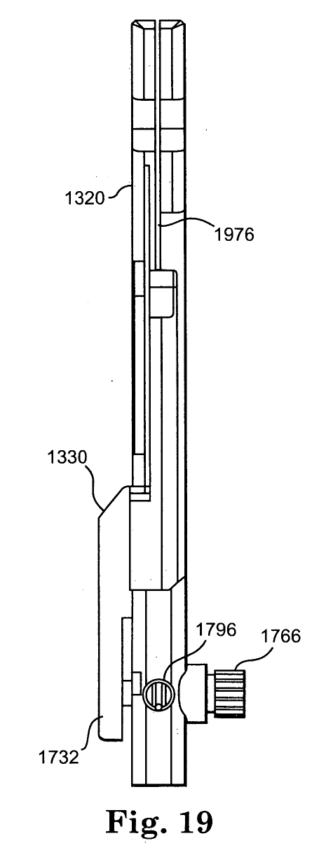

Referring to FIG. 17, 18 and 19, in a sixth embodiment of the invention, a cutter 1700 has a locking member 1790 that selectively maintains a switching member 1730 in either an open or closed position. The locking member 1790 in this case has a locking pin 1768 mounted on a base portion 1720. The switching member 1730 has an upper locking hole 1758 and lower locking hole 1760, into which the locking pin 1768 can be selectively inserted. The switching portion 1730 is pivotally attached to the base portion 1720 by a spring-loaded pivot connection that permits the switching member 1730 to shift sideways along the pivot axis. As best illustrated in FIG. 19, the switching member 1730 includes a side portion 1732, which is connected to a push button 1766 through a shaft (not shown) that passes through the base member 1720. A compression spring 1796 winding over the shaft biases the shaft such that the side frame 1732 is biased toward the base member 1720. Pushing the button 1766 forces the side frame 1732 to shift away from the base member 1720. The shaft is sufficiently long so that the side frame 1732 can pushed sufficiently far from the base member 1720 to be able to swing between the open and closed positions without being blocked by the locking pin 1768. A portion of the groove 1976 protecting the blade 1770 is sufficiently wide to permit the blade 1770 to shift when the side frame 1732 shifts away from the base member 1720.

In operation, when the switching member 1730 is to be positioned and locked in the open position to permit the removal or mounting of the blade 1770, the button 1766 is pushed to disengage the side frame 1732 from the locking pin 1768. The switching member 1730 is then rotated to the desired location (open or closed). The push button 1766 is then released. The side frame 1732 thus moves back toward the base member 1720 by the force of the compressive spring, engaging the locking pin 1768 inside one of the two locking holes 1758 and 1760. The upper locking hole 1758 locks the switching member 1730 in the closed position; the lower locking hole 1760 locks the switching member 1730 in the open position.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. A cutter, comprising:
an elongated body having a distal portion and a proximal portion;
a blade holder disposed at least partially in the distal portion and adapted to secure a blade having a cutting edge, the blade holder being movable between a withdrawn position and a advanced position;
a blade shield positioned to cover substantially the entire cutting edge when the blade holder is in the withdrawn position and expose at least a portion of the cutting edge when the blade holder is in the advanced position; and
a stop disposed in the distal portion and adapted to stabilize an object to be cut against the cutting edge while the blade holder moves from the withdrawn position toward the advanced position.

2. The cutter of claim 1, further comprising an actuator at least partially disposed in the proximal end of the body and operatively connected to the blade holder to move the blade holder between the withdrawn position and the advanced position.

3. The cutter of claim 1, wherein the distal portion of the body defines a gap having an open end and a closed end, the gap being positioned and configured to receive the object to be cut into the gap through the open end and position the object at least partially between the cutting edge of the blade and the stop.

4. The cutter of claim 3, wherein the blade holder is mounted on the body via a sliding contact with the body.

5. The cutter of claim 4, wherein the stop comprises a block disposed across the gap from the blade and adapted to buttress the object to be cut against the blade as the blade advances toward the block.

6. The cutter of claim 5, wherein the blade holder comprises:
a base member in sliding contact with the body; and
a switching member connected to the base member and movable between an open position and a closed position relative to the base member, wherein the blade holder is configured to permit the blade to be mounted on, and detached from, the blade holder when the switching member is in the open position and to maintain the blade in an operating position when the switching member is in the closed position.

7. The cutter of claim 6, further comprising first locking member configured to maintain the switching member in the closed position relative to the base member.

8. The cutter of claim 7, wherein the blade is mounted on the base member.

9. The cutter of claim 7, wherein the blade is mounted on the switching member.

10. The cutter of claim 9, wherein the locking member is further configured to maintain the switching member in the open position relative to the base member.

11. The cutter of claim 10, wherein the locking member comprises:
a protrusion connected to one of the base member and switching member, and
two recesses defined on the other one of the base member and switching member, each of the recesses configured to engage the protrusion,
wherein the switching member is maintained in the open position relative to the base member when the protrusion is engaged to one of the two recesses, and in the closed position relative to the base member when the protrusion is engaged to the other one of the two recesses.

12. The cutter of claim 11, wherein the protrusion comprises movable tip portion and a resilient member biasing the tip portion toward the recesse that the protrusion is engaged to.

13. The cutter of claim 2, wherein the actuator comprise a lever pivotally mounted on the body, one end of the level being adapted to receive a force applied by an operator of the cutter.

14. The cutter of claim 4, wherein the blade holder comprises an distal portion overhanging the body, and the cutter further comprises a guide member over hanging the body and, during at least a portion of the blade holder's advancement from the withdraw position to the advanced position, engages the distal portion of the blade holder and biases the distal portion of the blade holder towards the body.

15. A cutter, comprising:
an elongated body having a distal portion and a proximal portion;
a support mounted on the body and adapted to slide relative to the body;
a blade attached to the support and disposed at least partially in the distal portion and having a cutting edge, the blade being movable between a withdrawn position and a cutting position;
a blade shield positioned to cover substantially the entire cutting edge when the blade is in the withdrawn position and expose at least a portion of the cutting edge when the blade is in the cutting position; and
a stop disposed in the distal portion and adapted to stabilize an object to be cut against the cutting edge while the blade moves from the withdrawn position toward the cutting position.

16. The cutter of claim 15, wherein the distal portion of the body defines a gap having an open end and a closed end, the gap being positioned and configured to receive the object to be cut into the gap through the open end and position the object at least partially between the cutting edge of the blade and the stop.

17. The cutter of claim 16, wherein the distal portion of the body further comprises a protrusion adjacent the open end of the gap, the protrusion configured to maintain the object to be cut toward the closed end when the object is cut.

18. The cutter of claim 15, wherein the blade is manually detachable from the blade holder.

19. The cutter of claim 18, wherein the blade holder comprises:
a base member in sliding contact with the body; and
a switching member connected to the base member and movable between an open position and a closed position relative to the base member, wherein the blade holder is configured to permit the blade to be mounted on, and detached from, the blade holder when the switching member is in the open position and to maintain the blade in an operating position when the switching member is in the closed position.

20. The cutter of claim 19, further comprising a locking member configured to maintain the switching member selectively in the closed position and the open position relative to the base member.

21. A method of severing an object disposed in an incision in a patient, the method comprising:
mounting a blade on a blade holder of a cutter, the blade comprising a cutting edge, and the cutter comprising:
an elongated body having a distal portion and a proximal portion,
a blade holder disposed at least partially in the distal portion and adapted to secure the blade, the blade holder being movable between a withdrawn position and a advanced position,
a blade shield positioned to cover substantially the entire cutting edge when the blade holder is in the withdrawn position and expose at least a portion of the cutting edge when the blade holder is in the advanced position, and
a stop disposed in the distal portion and adapted to stabilize an object to be cut against the cutting edge while the blade holder moves from the withdrawn position toward the advanced position,
placing the blade holder in the withdrawn position;
while maintaining the blade holder in the withdrawn position, placing the distal end of the cutter inside the incision and positioning the object between the blade and the stop; and
moving the blade holder toward the advanced position to sever the object.

22. The method of claim 21, wherein positioning the object between the blade and the stop is carried out without handling the object by hand or any instrument other than the cutter.
